(19) United States
(12) Patent Application Publication
(10) Pub. No.: US 2026/0020989 A1
(43) Pub. Date: Jan. 22, 2026

(54) NASAL MUCUS CAPTURE DEVICE

(71) Applicant: Ramona Chairez, Fort Worth, TX (US)

(72) Inventor: Ramona Chairez, Fort Worth, TX (US)

(21) Appl. No.: 19/272,467

(22) Filed: Jul. 17, 2025

Related U.S. Application Data
(60) Provisional application No. 63/673,258, filed on Jul. 19, 2024.

(51) Int. Cl. A61F 13/45; A61F 13/15

(52) U.S. Cl. CPC A61F 13/45; A61F 2013/15008

(57) ABSTRACT

A simple, sanitary, disposable, and cost-effective device that allows a user to safely capture mucus from one's nose and dispose of such mucus in a sanitary manner. The nasal mucus capture device may include a first layer, a second layer secured to the first layer, and a third layer secured to the first and second layers, wherein the first, second, and third layers are in a substantially cylindrical configuration. In addition, a top region of the first and second layer can be disposed between the third layer, wherein the third layer may include a cotton pad.

### CROSS-REFERENCE TO RELATED APPLICATION

This application claims the benefit of U.S. Provisional Patent Application No. 63/673,258 filed on Jul. 19, 2024, which is incorporated herein by reference in its entirety.

### BACKGROUND

This section is intended to introduce the reader to aspects of art that may be related to various aspects of the present disclosure described herein, which are described and/or claimed below. This discussion is believed to be helpful in providing the reader with background information to facilitate a better understanding of the various aspects of the present disclosure described herein. Accordingly, it should be understood that these statements are to be read in this light, and not as admissions of prior art.

It is common for a user to use tissue paper, Q-tips, or cotton balls to clean, sanitize, or capture mucus from the user's nose. However, such devices and methods, such as tissue paper, do not allow for adequate and sanitary cleaning and capturing of nasal mucus. For example, if one blows and expels mucus into tissue paper, then it is likely that the tissue paper cannot capture the various nasal mucus particles and some particles may inevitably spread into the air or surrounding areas. Moreover, handling and disposing of the used tissue paper (with mucus deposited therein) can also be highly unsanitary, which may require the user to also wash his/her hands afterwards, which can be a further inconvenience. In other words, no device exists that allows a user to capture nearly all of the nasal mucus expelled from the nose and dispose of that captured nasal mucus in a sanitary and clean manner.

Hence, what is needed is a simple, sanitary, and cost-effective device that allows a user to safely capture nearly all of the mucus from one's nose and dispose of such mucus in a sanitary manner.

### BRIEF SUMMARY

In one aspect of the disclosure described herein, a simple, sanitary, disposable, and cost-effective device is disclosed that allows a user to safely capture nearly all or all of the mucus (or mucous) from one's nose and dispose of such mucus in a sanitary. A nasal mucus capture device may include a first layer, a second layer secured to the first layer, and a third layer secured to the first and second layers, wherein the first, second, and third layers are rolled into a substantially cylindrical configuration. In addition, a top region of the first and second layer may be disposed between the third layer. Further, the third layer may include a cotton pad. Also, the second layer may include a non-woven material. In addition, the nasal mucus capture device can include a fourth layer secured to a lower end of the rolled cylindrical configuration of first layers. Further, the second layer may be adapted to capture nasal mucus. Also, the third layer may be disposed over a top brim region of the rolled cylindrical configuration of the nasal mucus capture device. Moreover, the third layer may be scented.

Here, some advantages of the nasal mucus capture device disclosed herein can also include enhanced control over the cleaning process, less expelling and spread of mucus secretion and spreading of germs; less irritation from constant cleaning of the nose with handkerchief or paper, tissue and towels; the device can be made of recyclable material for one time use and also be environmental friendly; the device can prevent make up users from ruining the set, make up on the face; the device can also prevent people who use preventative products for facial irritation from being wiped away; the device can also prevent embarrassing nose blowing incidents where at times the mucus from runny nose or secretions can blow out of a tissue onto the face, leaving remnants of mucus on nose, face, beard, and even clothing; and a cotton tipped openings or distal end member can also allow for a quick cleaning around the inside of the nostril helping to clean any remnants of mucus once the nose has been blown; and the device of the disclosure described herein can prevent the user from touching or coming into contact with the mucus given that the mucus is contained within the device, among other advantages.

The above summary is not intended to describe each and every disclosed embodiment or every implementation of the disclosure. The Description that follows more particularly exemplifies the various illustrative embodiments.

### BRIEF DESCRIPTION OF THE DRAWINGS

The following description should be read with reference to the drawings, in which like elements in different drawings are numbered in like fashion. The drawings, which are not necessarily to scale, depict selected embodiments and are not intended to limit the scope of the disclosure. The disclosure may be more completely understood in consideration of the following detailed description of various embodiments in connection with the accompanying drawings, in which:

FIG. 1 illustrates a simplified cross-sectional side view of the nasal mucus capture device of the disclosure described herein, according to one or more non-limiting exemplary embodiments.

FIG. 2A illustrates top views for various layers of the nasal mucus capture device of the disclosure described herein, according to one or more non-limiting exemplary embodiments.

FIG. 2B illustrate a top view for a layer overlaid on another layer of the nasal mucus capture device of the disclosure described herein, according to one or more non-limiting exemplary embodiments.

FIG. 3A illustrates a perspective view for the nasal mucus capture device of the disclosure described herein, according to one or more non-limiting exemplary embodiments.

FIG. 3B illustrates another perspective view for the nasal mucus capture device of the disclosure described herein, according to one or more non-limiting exemplary embodiments.

FIG. 3C illustrates another perspective view for the nasal mucus capture

device of the disclosure described herein, according to one or more non-limiting exemplary embodiments.

FIG. 4 illustrates a perspective interior view for the nasal mucus capture device of the disclosure described herein, according to one or more non-limiting exemplary embodiments.

FIG. 5 illustrates another perspective interview for the nasal mucus capture device of the disclosure described herein, according to one or more non-limiting exemplary embodiments.

FIG. 6 illustrates a perspective exterior view for the nasal mucus capture device of the disclosure described herein, according to one or more non-limiting exemplary embodiments.

FIG. 7A illustrates a perspective side view for the nasal mucus capture device of the disclosure described herein, according to one or more non-limiting exemplary embodiments.

FIG. 7B illustrates a simplified cross-sectional side view for the various layers of the nasal mucus capture device of the disclosure described herein, according to one or more non-limiting exemplary embodiments.

FIG. 7C illustrates a simplified cross-sectional top view for the one or more layers of the nasal mucus capture device of the disclosure described herein, according to one or more non-limiting exemplary embodiments.

FIG. 8 illustrates a perspective top view for a container or packaging for

multiple nasal mucus capture devices of the disclosure described herein, according to one or more non-limiting exemplary embodiments.

FIG. 9 illustrates a perspective view for another embodiment of the nasal mucus capture device of the disclosure described herein, according to one or more non-limiting exemplary embodiments.

### DETAILED DESCRIPTION

In the Brief Summary of the present disclosure above and in the Detailed Description of the disclosure described herein, and the claims below, and in the accompanying drawings, reference is made to particular features (including method steps) of the disclosure described herein. It is to be understood that the disclosure of the disclosure described herein in this specification includes all possible combinations of such particular features. For example, where a particular feature is disclosed in the context of a particular aspect or embodiment of the disclosure described herein, or a particular claim, that feature can also be used, to the extent possible, in combination with and/or in the context of other particular aspects and embodiments of the disclosure described herein, and in the disclosure described herein generally.

The embodiments set forth below represent the necessary information to enable those skilled in the art to practice the disclosure described herein and illustrate the best mode of practicing the disclosure described herein. In addition, the disclosure described herein does not require that all the advantageous features and all the advantages need to be incorporated into every embodiment of the disclosure described herein.

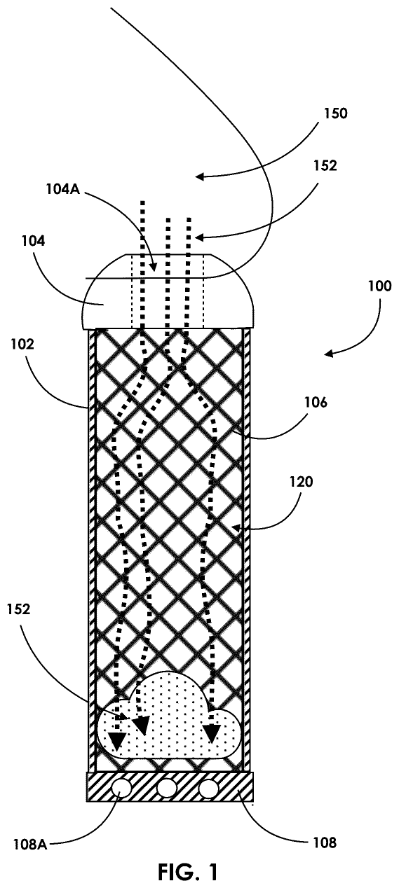

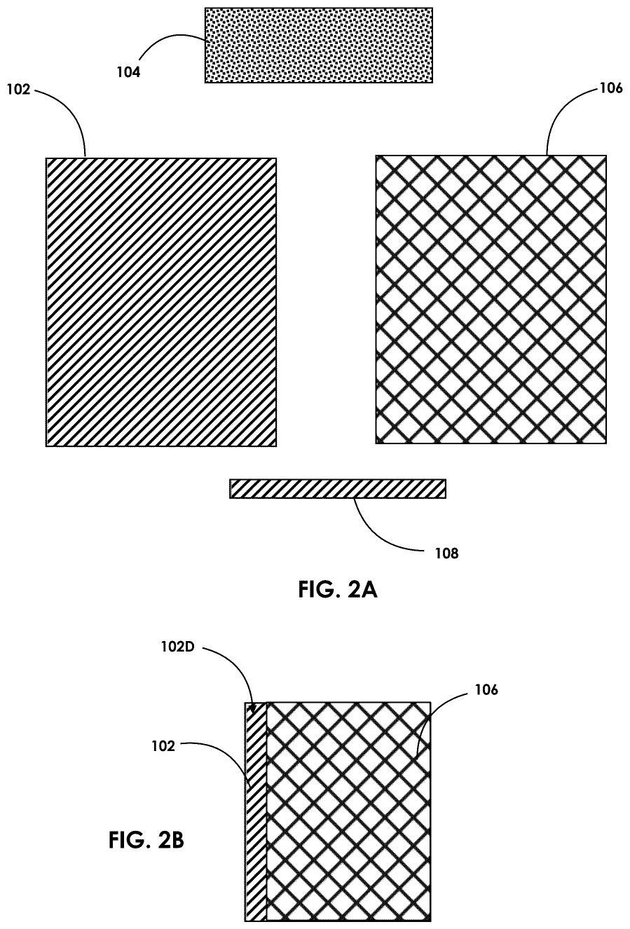

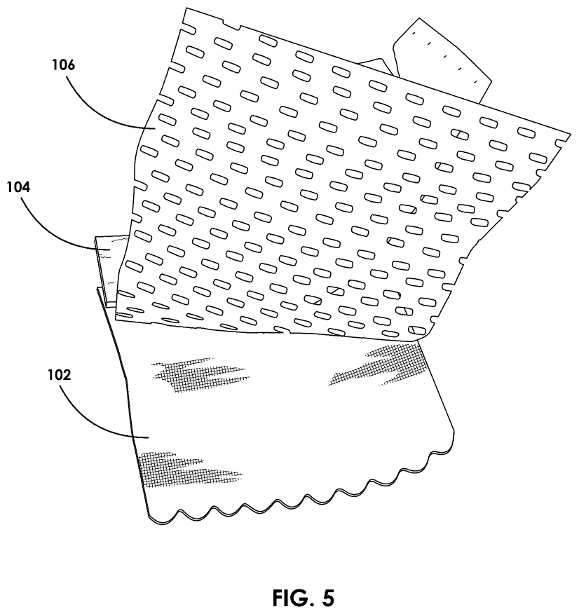

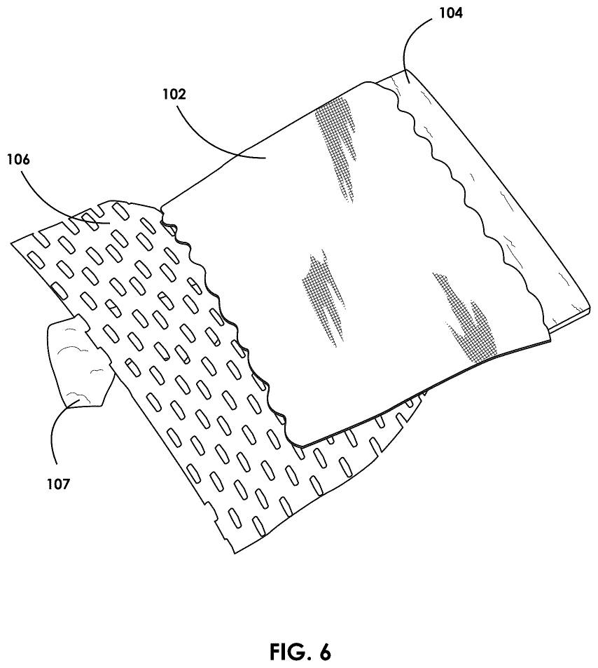

FIGS. 1-7C illustrate one or more non-limiting exemplary embodiments of the nasal mucus capture device 100 of the disclosure described herein. As shown, device 100 can include a main outer layer or housing 102 that can be wrapped, rolled, or configured in a cylindrical tube. Here, layer or housing 102 can be made of any fiber, cellulose fibers, wood pulp, paper, cardboard, card stock, cotton, woven or non-woven material, plastic, recyclable plastic or material, silicone, metal, wood, rubber, hydrophobic fabric, polypropylene material, or the like. However, it is contemplated within the scope of the present disclosure described herein that layer 102 may be made of any material. In some embodiments, layer 102 can be semi-rigid and flexible, and in other embodiments, layer 102 can be rigid. In some embodiments, layer 102 can include a structure or a thickness that would prevent a collapse of the walls while device 100 is being used or handled by a user. In some embodiments, layer 102 may be anywhere from about 1 in. up to about 5 in. in length, or preferably about 2.1 inches in length, and from about 0.5 in up to about 6 in. in width or height, or preferably about 1.4 inches in width or height, and about 0.005 in. to about 1 in. in thickness.

Still referring to FIGS. 1-7C, device 100 can also include inner layer or pad 106 disposed or secured to the top of layer 102, wherein layer 106 may be wrapped, rolled, or configured in a cylindrical tube within layer 102. Here, layer 106 can be made of any fiber, cotton, gauze, air/mucus filtering medium, tissue, 1-ply or 2-ply or 3-ply or 4-ply non-woven or woven fabric, cellulose fibers, hydrophobic fabric, polypropylene material, among others. However, it is contemplated within the scope of the present disclosure described herein that layer 106 may be made of any material. In addition, layer 104 may be odorless and have various anti-bacterial or anti-viral properties or additives. In some embodiments, layer 106 can be flexible to allow for easy rolling while preventing the walls to collapse in a rolled and/or assembled configuration. In some embodiments, layer 106 may be anywhere from about 1 in. up to about 5 in. in length, or preferably about 2.0 inches in length, and about 0.5 in to about 6 in. in width or height, or preferably about 1.4 in, and a thickness of about 0.005 to about 1 in. Here, it is noted that layer 106 may be slightly shorter in length (such as by a few-tenths of an inch) than layer 102, such that layer 106 can effectively wrap over and envelope layer 106 within. In particular, in assembled form, layer 106 have one edge aligned with an edit of layer 102, such an inner end area or surface 102D of layer 102 is exposed for allowing it to effectively adhere to the exterior surface of layer 102 when rolled into its assembled configuration.

Still referring to FIGS. 1-7C, device 100 may also include a top layer or pad 104, wherein layer 104 may be wrapped, rolled, or configured in a substantially cylindrical tube within and over layer 102. Here, layer 104 can be made of any fiber, cotton, gauze, air/mucus filtering medium, tissue, 1-ply or 2-ply or 3-ply or 4-ply non-woven or woven fabric, hydrophobic fabric, polypropylene material, among others. However, it is contemplated within the scope of the present disclosure described herein that layer 104 may be made of any material. In some embodiments, layer 104 can be flexible to allow for easy rolling while preventing the walls from collapsing in a rolled and/or assembled configuration. In addition, layer 104 may be odorless and have various anti-bacterial or anti-viral properties or additives. Further, in some embodiments, device 100 may also include a bottom layer 108 that may be cut in a substantially round configuration and adapted to provide cap, cover, or floor to the bottom of device 100. Here, layer 104 may be made of any material such as any fiber, cotton, gauze, air/mucus filtering medium, tissue, 1-ply or 2-ply or 3-ply or 4-ply non-woven or woven fabric, cellulose fibers, wood pulp, paper, cardboard, card stock, cotton, or the like. However, it is contemplated within the scope of the present disclosure described herein that layer 104 may be made of any material. In some embodiments, layer 104 may be anywhere from about 1 in. up to about 5 in. in length, or preferably about 2.0 inches in length, and about 0.1 in to about 3 in. in width or height, or preferably about 0.5 in, and a thickness of about 0.005 in. to about 1 in. Generally, it is desirable for layer 104 to be the same or substantially the same length of layer 102, such that they can wrap over layer 106, but of shorter height or width such that it generally covers the top lip or brim of device 100.

As shown in FIG. 1, in one method of operation, a user can first slightly insert the top layer 104 into one nostril 150 of their nose (or at least create a seal between the opening of the nostril and the opening or channel 104A of layer 104 of device 100). Next, once layer 104 is securely fit within the nostril (or pressed against it such that the nostril opening and opening 104A are aligned), then the user press against the opposing nostril (or plug it) such that no air or mucus expulsion can occur from the opposing nostril. Here, it is desirable for the user to maintain a fitted or tight seal between member 104 and the circumference of the nostril in order to prevent any mucus from being expelled outside of device 100. Next, the user can then blow his/her nose, perform epistaxis, or expel mucus 152 into opening 104A and further into chamber 120 of device 100 for a desired amount of time and/or any number of times until the user feels that he/she has sufficiently cleared the nostril or his/her sinuses from any nasal mucus, such that mucus is collected and captured via fibers of both layers 104 and 106 of device 100. Next, the user can repeat the foregoing method with the opposing nostril in order to also clear that nostril. In addition, any excess mucus may be captured on the bottom floor or at the base of device 100. In some embodiments, the housing of device 100 (such as layers 102 and/or 106) that supports chamber 120 can also be clear, translucent, or semi-transparent such that the user can visually see how much mucus is captured or deposited within chamber 120. In other embodiments, layer 102 may also be opaque or set in a desired color. Alternatively, in some embodiments, device 100 may be configured without a bottom layer 108 such that is open at its base to allow expelled air to easily pass through, while still capturing mucus 152 within chamber 120. In other embodiments, bottom layer 108 may include a plurality of apertures 108A in fluid communication within chamber 106 and opening 104A in order to allow air dispensed from the user's nose to pass through and out of device 100, while still filtering and/or capturing any mucus within device 100.

Here, through various types of experimental testing, device 100, including the disclosed layers, sizing, and assembly, was found to be far more effective in capturing nasal mucus relative to conventional tissue paper, Q-tips, cotton balls, paper towels, handkerchiefs, among others. In fact, through such testing, users who were wearing make-up, foundation, lipstick, sunscreen, or any other facial beauty related products on their face and nose, were able to completely capture all nasal mucus while preventing any disruption or “smearing” of such products on the user's face relative to conventional tissue paper, Q-tips, cotton balls, paper towels, or handkerchiefs. Through such testing, it was found that conventional tissue paper, paper towels, Q-tips, cotton balls, or handkerchiefs wrapped or pressed against one's nose (from the outside) can inadvertently “smear”, remove, or disrupt such facial products applied to the face due to the large surface areas that such tissue paper, or in the case of Q-tips, the small area they can cover. In contrast, device 100 (given that it can be inserted within the nostril or around the nostril) prevents any direct contact with the outer nose surface, lips, cheeks, or chin regions of the user, which is highly advantageous and beneficial to users and prevents the user from having to reapply or “touch up” their make-up after simply blowing their nose relative to using conventional tissue paper, Q-tips, cotton balls, paper towels, handkerchiefs, etc.

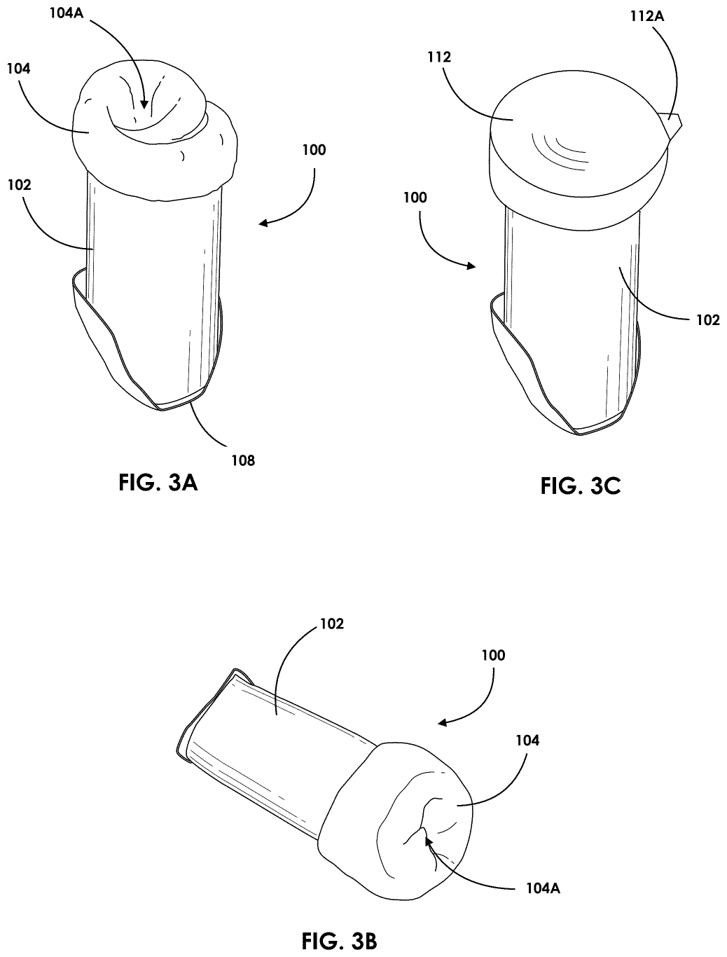

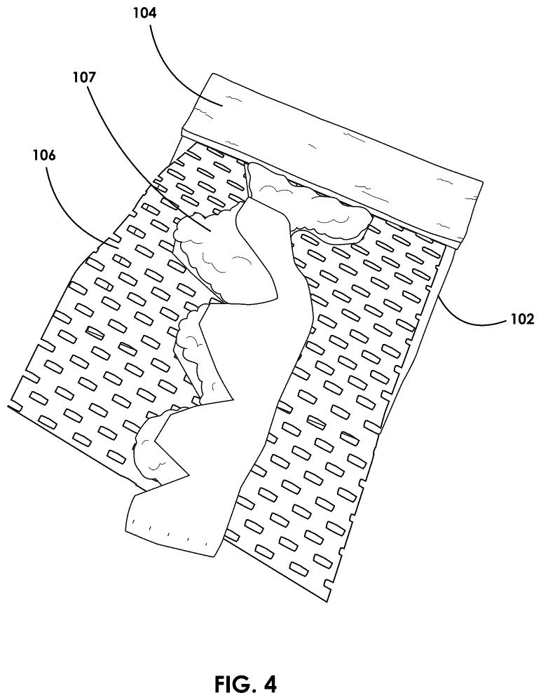

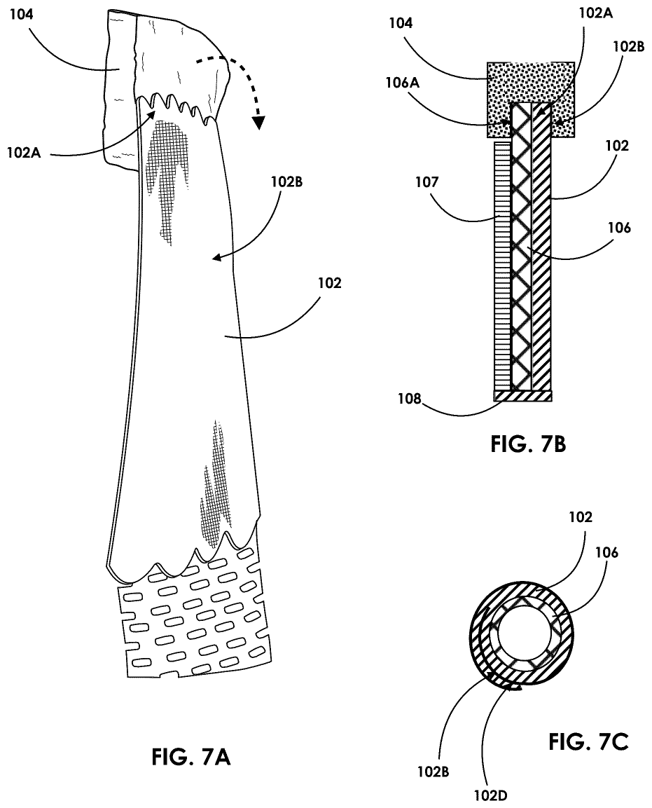

FIGS. 3A-7C further illustrate various embodiments for assembling, making, and/or manufacturing device 100 of the disclosure described herein. As shown, device 100 may have layer 102 pre-cut to the desired size and laid flat on a surface. Next, layer 106 may then be placed on top of layer 102 and secured to layer 102 via one or more adhesives (or stitching, hot pressing, hot glue, or any securement member or method) along its top surface region 102A (FIG. 7B). Alternatively, one or more adhesives may be applied to substantially the entire length of surface 102A of layer 102 (or bottom surface of layer 106). Optionally, in other embodiments, another layer 107 (FIG. 4) may also be secured to the top of surface 106, wherein layer 107 may also be another material such as any fiber, cotton, gauze, air/mucus filtering medium, tissue, 1-ply or 2-ply or 3-ply or 4-ply non-woven or woven fabric, cellulose fibers, wood pulp, paper, cardboard, card stock, cotton, or the like. Layer or pad 107 can further assist layer 106 to capture additional nasal mucus and may further be cut to various configurations to create baffles or vanes within chamber 120 (FIG. 1) of device 100. Next, layer 104 can then be placed on top of layer 106 and secured to the top surface layer 106A (FIG. 7B) of layer 106 via one or more adhesives (or stitching, hot pressing, hot glue, or any securement member or method) and then folded over (FIG. 7A-7B) to have an opposing side of layer 104 secured to the bottom surface 102B of layer 102 via one or more adhesives (or stitching, hot pressing, hot glue, or any securement member or method), such that the top regions of layers 106 and 102 are disposed and secured (i.e., sandwiched) between layer 104. Optionally, in other embodiments, while not shown, layer 104 may also be secured to the top of layer 107 vie one or more adhesives (or stitching, hot pressing, hot glue, or any securement member or method), such that the upper regions of layers 107, 106, and 102 are disposed and secured (i.e., sandwiched) between layer 104.

Still referring to FIGS. 3A-7C, once the various layers are secured to each other, then device 100 may be rolled into a substantially cylindrical tube, such that the tube may have a diameter of about 0.1 in. to about 3 in. FIG. 7C illustrates layer 102 (with the other layers omitted for illustration purposes) rolled into a substantially cylindrical tube wherein outer surface 102B can include an adhesive (or stitching, hot pressing, hot glue, or any securement member or method), such as at end region 102D, in order secure layer 102 into a roll. In some embodiments, it is noted that the inner end or edge region 102D of layer 102 that is exposed (not secured to layer 106), given that in some embodiments layer 102 is slightly longer in length than layer 106, that is secured to the outer surface of layer 102. Next, optionally, in other embodiments, layer 108 may be cut into a similar cross-sectional shape as that rolled and assembled device 100 and secured to the bottom or base of layer 102, such as shown in FIGS. 3A-3B and 7B. In addition, as shown in FIG. 3C, in other embodiments, each device 100 may also include a seal, cap, or cover 112 to cover layers 104 and opening 104A for sanitation purposes, among others. Here, cap 112 may be secured via one or more removable adhesives to the top of device 100, or in some embodiments, extend to the outer surface of layer 102. In addition, cap 112 may also include a tab 112A allowing it to be easily removed from device 100.

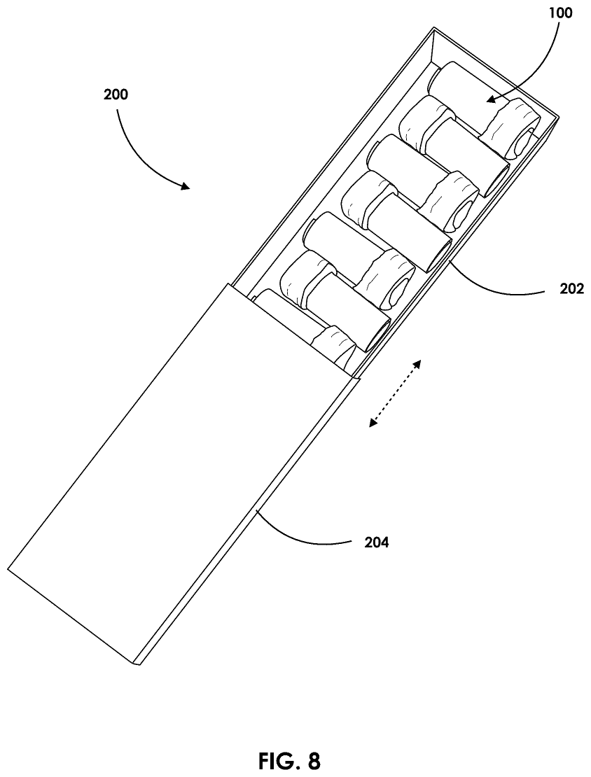

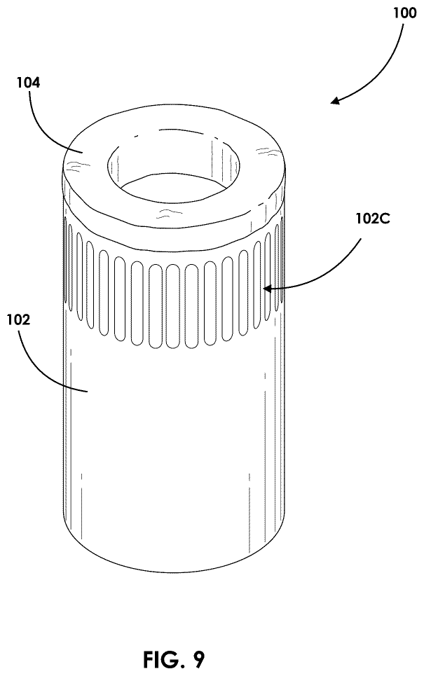

FIG. 8 illustrates one non-limiting exemplary embodiment for packaging devices 100. In particular, devices 100 may be stacked next to each other in container 200, wherein each is inverted relative to an adjacent device 100. Here, container 200 may include a matchbox style container having a drawer section 202 that can be pulled in and out of a sleeve 204. Here, container 200 can be sized in various configurations and sizes, such as sizes that can be easily placed inside of bags or purses or within a user's pocket, among other variations, and can be configured as a 2-pack, 3-pack, 4-pack, 5-pack, 6-pack, 7-pack, 8-pack, 9-pack, 10-pack, 11-pack, 12-pack devices 100, or anywhere from a 13 to 1000 pack devices 100 containers. FIG. 9 illustrates another non-limiting exemplary embodiment of device 100, shown with layers 102 and top layer 104 on the top brim of device 100, wherein layer 102 may also include ridges, indentations, or protrusions 102C on its exterior to assist the user with gripping device 100.

It is contemplated within the scope of the disclosure described herein, that the main chamber 120 of device 100 can also include a filter configured as any type of corrugated, baffled, cross-hatched, or cellulose type material adapted to capture additional viscous liquid nasal mucus material or deposits but allowing air expelled from the nose to pass through. Further, while device 100 is shown configured in a substantially cylindrical in configuration, it is contemplated within the scope of the present disclosure described herein that housing 102 may have any shape, size, or dimension, such as a square, ellipsoid, oval, or asymmetric configuration.

In addition, while device 100 is configured to be disposable or as one-time use, in other embodiments, device 100 may also be re-used such as allowing the chamber 120 of housing to be cleaned and/or having a filter within chamber 120 replaced. As an example, in one embodiment, base 108 may be removably threaded or secured onto the lower region of device 100, which would allow base 108 to be removed in order to clean or replace the filter within chamber 120 (and inserted back in) and base 108 then placed and threaded/secured back onto device 100. In addition, in other embodiments, one or more removable layers of cotton pads may be secured to the bottom of housing 100 or base 108, wherein each cotton pad layer can be removed in order to expose a clean unused cotton pad layer there below.

Further, it is also contemplated within the scope of the present disclosure described herein that in some embodiments, device 100 may not be rolled up as disclosed. For example, layer 102 (including any one or more of layers 104, 106, 107, and 108) may be a one-piece component formed in a continuous configuration having no seams and in a cylindrical or tubular configuration, such as via injection molding, stamping, or other methods, such as shown with respect to device 100 of FIG. 9. In addition, it is contemplated within the scope of the present disclosure described herein that device 100, such as layer 104 may be unscented or scented, such as having a eucalyptus, menthol or any type of pleasant essential oil type scent. In addition, each device 100 may also be individually wrapped in a wrapper or shrink wrapped.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts described herein, except insofar as such limitations are included in following claims. Further, it will be understood that certain features and sub-combinations are of utility and may be employed without reference to other features and sub-combinations. This is contemplated by and is within the scope of the claims.

What is claimed is:

1. A nasal mucous capture device, comprising:
a first layer;
a second layer secured to the first layer; and
a third layer secured to the first and second layers, wherein the first, second, and third layers are in a substantially cylindrical configuration.

2. The nasal mucus capture device of claim 1, wherein a top region of the first and second layer is disposed between the third layer.

3. The nasal mucus capture device of claim 2, wherein the third layer is comprised of a cotton pad.

4. The nasal mucus capture device of claim 3, wherein the second layer is comprised of a non-woven material.

5. The nasal mucus capture device of claim 1, further comprising a fourth layer secured to a lower end of the cylindrical configuration of the first layer.

6. The nasal mucus capture device of claim 4, wherein the second layer is adapted to capture nasal mucus.

7. The nasal mucus capture device of claim 1, wherein the third layer is disposed over a top brim region of the cylindrical configuration of the nasal mucus capture device.

8. The nasal mucus capture device of claim 1, wherein the third layer is scented.
